(19) United States
(12) Patent Application Publication
(10) Pub. No.: US 2008/0039424 A1
(43) Pub. Date: Feb. 14, 2008

(54) Cosmetic composition comprising at least one polysaccharide chosen from lambda-carrageenans in combination with at least one non-ionic surface-active agent chosen from alkylpolyglucosides, and a method for the cosmetic treatment of keratinous fibers therewith

(76) Inventors: Serge Restle, Saint Prix (FR); Ludivine Laurent, Courbevoie (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/882,778

(22) Filed: Aug. 6, 2007

Related U.S. Application Data
(60) Provisional application No. 60/855,739, filed on Nov. 1, 2006.

(30) Foreign Application Priority Data
Aug. 4, 2006 (FR) 06/07158

(51) Int. Cl. A61K 31/70; A61P 17/00

(52) U.S. Cl. 514/54

(57) ABSTRACT

The present disclosure relates to a cosmetic composition for the treatment of keratinous fibers, such as the hair, comprising at least one polysaccharide chosen from lambda-carrageenans and at least one non-ionic surface-active agent chosen from alkylpolyglucosides, wherein the at least one polysaccharide is present in an amount ranging from 0.1 to 30% by weight of the total weight of the composition. The present disclosure also relates to a cosmetic method for treating keratin fibers and a cosmetic method for fixing and caring for the fibers.

This application claims benefit of U.S. Provisional Application No. 60/855,739, filed Nov. 1, 2006, the contents of which are incorporated herein by reference. This application also claims benefit of priority under 35 U.S.C. § 119 to French Patent Application No. FR 06/07158, filed Aug. 4, 2006, the contents of which are also incorporated herein by reference.

The present disclosure relates to a cosmetic composition for the treatment of keratinous fibers, for instance human keratinous fibers, such as the hair, comprising at least one polysaccharide chosen from lambda-carrageenans and at least one specific surface-active agent, and also to a cosmetic treatment method for fixing or caring for the said fibers.

In the styling field, such as among hair products intended for shaping and/or retaining the hairstyle, hair compositions may be composed of a solution, for instance, an alcoholic and/or aqueous solution, and of at least one fixing polymer as a mixture with various cosmetic adjuvants.

These compositions can be provided in the form of hair gels or mousses which may be applied to wet hair before blow drying or drying. Hair gels may be composed, for example, of at least one thickening polymer or gelling agent in combination with at least one fixing polymer, which generally have the role of forming a film at the surface of the keratinous fibers to be fixed.

Recently, carrageenans have been used as fixing polymers in styling gels or in aerosols. For example, it is known from European Patent Application No. EP-A-1 199 064 to employ a carrageenan or a mixture of the latter and a specific additive in order to obtain a solid and stable gel for hair treatment.

Carrageenans are polysaccharides which form the cell walls of various red algae (Rhodophyceae) belonging to the families of the Gigartinaceae, Hypneaceae, Furcellariaceae and Polyideaceae. They comprise long galactan chains which are anionic polyelectrolytes. Their molecular weights can be greater than 10⁶ a.m.u. (atomic mass unit). These linear polymers, formed by disaccharide units, are composed of two D-galactopyranose units alternately bonded via α- and β-bonds. These are highly sulphated (20-50%) polysaccharides and the α-D-galactopyranosyl residues can be in the 3′,6′-anhydro form.

The carrageenans were initially subdivided into two families according to their solubility in potassium chloride (KCl). The fractions soluble in KCl were designated by the prefixes “kappa”, while the terms “lambda” were reserved for those which are insoluble. Later, the classifications were based on the number of sulphate groups, the position of sulphate groups and the presence of 3′,6′-anhydro bridge on the β-D-galactopyranosyl residues. This resulted in four main families: κ, λ, β and ω.

The various types of carrageenans do not exist in the pure state but in the form of hybrids. Thus, in the natural state, the κ- and ι-carrageenans exist in a kappa-iota hybrid form but one of the two structures may predominate over the other. The κ-ι hybrid state of a structure can be elucidated using specific enzymes which make it possible to enrich or reduce the content of one of the two forms. The carrageenans can coexist with their precursors. The carrageenans belonging to different families can coexist in a hybrid structure, for example: carrageenan from Euchema gelatinae, a hybrid of β-carrageenan, major component, and of κ- and γ-carrageenans.

As described in European Patent Application No. EP-A-0 445 659, it is known to combine carrageenans with a specific oxyethylenated non-ionic surface-active agent with the aim of obtaining a specific adhesive strength between the hairs, of producing a soft and elastic film, of producing a softness effect on dry hair, and of maintaining the shape of the hair. However, the compositions described do not make it possible to obtain a satisfactory treating effect on wet hair before blow drying.

Likewise, it is known, from Japanese Patent Application No. JP-A-05-178718, to combine a carrageenan powder, the particles of which have a defined size, with a cosmetic additive, such as a surfactant, with the aim of obtaining, after expansion of the powder, a composition having a soft feel and a non-tacky effect. This document discloses, for instance, the use of kappa-carrageenan.

Moreover, a composition comprising λ-carrageenan without a mousseing agent does riot make it possible to obtain a styling mousse with a satisfactory texture.

There thus exists a real need to find cosmetic compositions, for example for hairstyling, which make it possible to overcome one or more of the abovementioned disadvantages.

Accordingly, the present inventors have discovered, surprisingly and advantageously, that, by combining at least one λ-carrageenan with at least one alkylpolyglucoside, it is possible to obtain a hair composition having good care properties on application to wet hair while retaining excellent styling and cosmetic properties on dried hair. Furthermore, the use of this λ-carrageenan in such a combination makes it possible to obtain gels; with better textures which are less liable to break down, which are softer and/or which are easier to apply.

Furthermore, in the aerosol form, this combination makes it possible to obtain a styling mousse with a satisfactory texture.

One aspect of the present disclosure, therefore, is a cosmetic composition for the treatment of keratinous fibers, e.g., human keratinous fibers such as the hair, comprising, in a cosmetically acceptable medium:

at least one polysaccharide chosen from lambda-carrageenans, and

at least one non-ionic surface-active agent chosen from alkylpolyglucosides.

Another aspect of the present disclosure relates to a cosmetic composition for the treatment of keratinous fibers, for example human keratinous fibers, such as the hair, comprising, in a cosmetically acceptable medium,

at least one polysaccharide chosen from lambda-carrageenans, and

at least one non-ionic surface-active agent chosen from alkylpolyglucosides,

wherein the at least one polysaccharide chosen from lambda-carrageenans is present in an amount between 0.1 and 30% by weight of the total weight of the composition.

The present disclosure further relates to a cosmetic method for treating keratin fibers employing the cosmetic composition disclosed herein.

The present disclosure also relates to a cosmetic method of caring or fixing the keratinous fibers, such as the hair, comprising applying the cosmetic composition disclosed herein to the hair.

Other benefits, characteristics, and aspects of the disclosure will become even more clearly apparent upon reading the description and examples which follow.

As used herein, the term “styling” is understood to mean the fact of fixing and/or of retaining the form of the hairstyle.

The composition disclosed herein comprises at least one polysaccharide chosen from lambda-carrageenans. In at least one embodiment, the polysaccharide chosen from lambda-carrageenans used according to the present disclosure is not chemically modified.

The molecular weight (MW) of the polysaccharide, in at least one embodiment, ranges from 100,000 to 1,000,000 a.m.u., such as, for example, between 250,000 and 800,000 a.m.u.

Non-limiting mention of the polysaccharide chosen from lambda-carrageenans which can be used in the context of the present disclosure include Satiagum UTC 10 from Degussa and Welgeenan ED 1039 from Eurogum.

As used herein, the term “between” is understood to mean that the values at the limits are excluded from the range of values described.

In at least one embodiment, the polysaccharide chosen from lambda-carrageenans can be present in the composition in an amount ranging from 0.2 to 20% by weight, and, for example, between 0.5 and 15% by weight, with respect to the total weight of the cosmetic composition.

The non-ionic surface-active agents chosen from alkylpolyglucosides which can be used in the presently disclosed composition are well known products which can be represented, for example, by the following formula (I):
R₁—O—(R₂—O)ₐ-(L)_{b} (I)

wherein:

R₁ is chosen from linear or branched alkyl and/or alkenyl radicals comprising from 8 to 24 carbon atoms and from alkylphenyl radicals, the linear or branched alkyl group of which comprises from 8 to 24 carbon atoms,

R₂ is chosen from alkylene radicals comprising from 2 to 4 carbon atoms,

L is chosen from reduced sugars comprising from 5 to 6 carbon atoms,

a ranges from 0 to 10, and

b ranges from 1 to 15.

In at least one embodiment, the alkylpolyglucosides disclosed herein are compounds of formula (I) wherein:

R₁ is chosen from linear or branched alkyl and/or alkenyl radicals comprising from 9 to 14 carbon atoms,

a is an integer ranging from 0 to 3 and, in at least one embodiment, equal to zero, and

L is chosen from glucose, fructose and galactose, and in at least one embodiment, L is glucose.

The degree of polymerization (S) of the saccharide, i.e. the value of b in the formula (I), can range from 1 to 15. For example, the reduced sugars comprising 80% or more of sugars for which the degree of polymerization (S) takes a value ranging from 1 to 4 may be used.

Compounds of formula (I) are represented, for example, by the products sold by Henkel under the name APG, such as the products APG 300, APG 350, APG 500, APG 550, APG 625 and APG base 10-12; the products sold by Seppic under the names Triton CG 110 (or Oramix CG 110) and Triton CG 312 (or Oramix NS 10); those sold by B.A.S.F. under the name Lutensol GD 70; and those sold by Henkel under the names Plantaren 1200, Plantaren 1300, Plantaren 2000, Plantacare 2000, Plantacare 818 and Plantacare 1200.

The alkylpolyglucoside(s) can be present in the composition in an amount ranging from 0.05 to 30% by weight, such as between 0.1 and 15% by weight, of the total weight of the cosmetic composition.

The cosmetic compositions in accordance with the present disclosure can be provided in a cream, mousse (aerosol or non-aerosol), paste or gel form. For example, this gel exhibits a viscosity of greater than 200 cPs at 25° C. at a shear rate of 1 s⁻¹. The viscosity ranges, further for example, from 500 to 500,000 cPs at 25° C., such as between 500 and 100,000 cPs at 25° C. and, even further for example, between 500 and 50,000 cPs at 25° C., at a shear rate of 1 s⁻¹. It can be measured using a viscometer of cone/plate type.

The composition according to the present disclosure can also be washing or non-washing and can comprise a washing base known to a person skilled in the art.

As disclosed herein, the composition can additionally comprise optional components.

The cosmetic composition according to the present disclosure can, further, additionally comprise at least one silicone.

As used herein, the term “silicone” is understood to mean any organosilicon polymer or oligomer with a linear or cyclic, branched or crosslinked structure, of variable molecular weight, obtained by polymerization and/or by polycondensation of suitably functionalized silanes and comprising a repetition of main units wherein the silicon atoms are connected to one another via oxygen atoms (siloxane —Si—O—Si— bond), optionally substituted hydrocarbon radicals being directly connected via a carbon atom to the silicon atoms. The hydrocarbon radicals can be, for instance, alkyl radicals, such as C₁-C₁₀ alkyl radicals, e.g., methyl radicals; fluoroalkyl radicals, the alkyl part of which is a C₁-C₁₀ alkyl radical; or aryl radicals, for example, a phenyl radical.

In one embodiment, the silicone is an oxyalkylene silicone.

As used herein the term “oxyalkylene silicone” is understood to mean any silicone comprising at least one oxyalkylene group of (—CxH2xO—)ₐ type wherein x can vary from 2 to 6 and a is greater than or equal to 2.

The oxyalkylene silicones which can be used in the cosmetic composition can be chosen from the following formulae (VI), (VII), (VIII) or (IX): 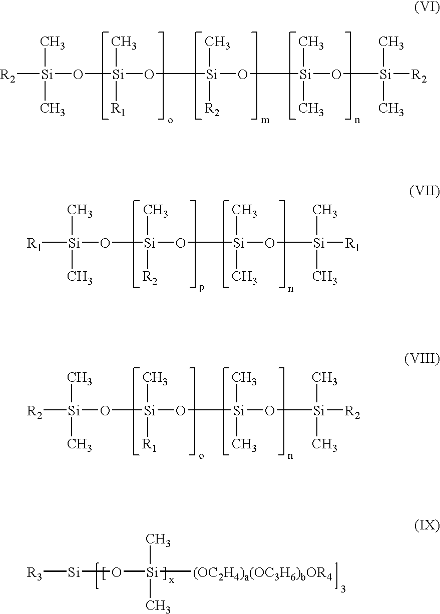
wherein:

R₁, which are identical or different, are chosen from linear or branched C₁-C₃₀ alkyl radicals or phenyl radicals,

R₂, which are identical or different, are chosen from —C_{c}H_{2c}—O—(C₂H₄O)ₐ(C₃H₆O)_{b}—R₅ radicals and —C_{c}H_{2c}—O—(C₄H₈O)ₐ—R₅ radicals,

R₃ and R₄, which are identical or different, are chosen from linear or branched C₁ to C₁₂ alkyl radicals, for example, methyl radicals,

R₅, which are identical or different, are chosen from hydrogen, linear or branched alkyl radicals comprising 1 to 12 carbon atoms, linear or branched alkoxy radicals comprising 1 to 6 carbon atoms, linear or branched acyl radicals comprising 2 to 30 carbon atoms, hydroxyl radicals, —SO₃M radicals, C₁-C₆ aminoalkoxy radicals optionally substituted on the amine, C₂-C₆ aminoacyl radicals optionally substituted on the amine, —NHCH₂CH₂COOM radicals, —N(CH₂CH₂COOM)₂ radicals, aminoalkyl radicals optionally substituted on the amine and on the alkyl chain, C₂-C₃₀ carboxyacyl radicals, groups optionally substituted by one or two substituted aminoalkyl radicals, —CO(CH₂)_{d}COOM groups, —COCHR₇(CH₂)_{d}COOM groups, —NHCO(CH₂)_{d}OH groups, —NH₃Y groups, and phosphate groups,

M, which are identical or different, are chosen from hydrogen, Na, K, Li, NH₄, and organic amines,

R₇ is chosen from hydrogen and SO₃M radicals,

d ranges from 1 to 10,

m ranges from 0 to 20,

n ranges from 0 to 500,

o ranges from 0 to 20,

p ranges from 1 to 50,

a ranges from 0 to 50,

b ranges from 0 to 50,

a+b is greater than or equal to 2,

c ranges from 0 to 4,

x ranges from 1 to 100,

Y⁻ is chosen from monovalent inorganic or organic anions, such as halide (chloride, bromide), sulphate or carboxylate (acetate, lactate, or citrate),

with the proviso that, when the silicone is of formula (VII) with R₅ being chosen from hydrogen, then n is greater than 12.

Such silicones are, for example, sold by Goldschmidt under the tradenames Abil WE 09, Abil EM 90, Abil B8852, Abil B8851, Abil B 8843, or Abil B8842, by Dow Corning under the names Fluid DC 190, DC 3225 C, Q2-5220, Q2-5354, or Q2-5200, by Rhodia Chimie under the names Silbione Oil 70646 or Rhodorsil Oil 10634, by General Electric under the names SF1066 or SF1188, by SWS Silicones under the name Silicone Copolymer F 754, by Amerchol under the name Silsoft Beauty Aid SL, by Shin-Etsu under the name KF 351, by Wacker under the name Belsil DMC 6038, by Siltech under the names Silwax WD-C, Silwax WD-B, Silwax WD-IS, Silwax WSL, Silwax DCA 100, or Siltech Amine 65, by Fanning Corporation under the names Fancorsil SLA or Fancorsil LIM1 or by Phoenix under the name Pecosil.

These silicones are described, for example, U.S. Pat. Nos. 5,070,171, 5,149,765, 5,093,452, and 5,091,493.

In at least one embodiment of the present disclosure, the polyoxyalkylene silicones corresponding to the formulae (VII) and (VIII) may be used. For instance, these formulae can correspond to at least one of the following conditions and/or all the following conditions:

c is equal to 2 or 3,

R₁ is chosen from methyl radicals,

R₅ is chosen from methyl radicals, C₁₂-C₂₂ acyl radicals, and —CO(CH₂)_{d}COOM radicals, d and M being as defined above,

a ranges from 2 to 25, for example, from 2 to 15,

b is equal to 0,

n ranges from 0 to 100,

p ranges from 1 to 20.

The polyoxyalkylene silicones can also be chosen from the silicones comprising at least one unit of following formula (X):
([Z(R₂SiO)_{q}R′₂SiZO][(CₙH₂ₙO)ᵣ])ₛ (X)
wherein:

R and R′, which are identical or different, are chosen from monovalent C₁-C₃₀ hydrocarbon radicals,

n is an integer ranging from 2 to 4,

q is an integer greater than or equal to 4, such as from 4 to 200 and, further for example, from 4 to 100,

r is an integer greater than or equal to 4, such as from 4 to 200 and, further for example, between 5 and 100,

s is an integer greater than or equal to 4, such as from 4 to 1,000 and, further for example, between 5 and 300,

Z, which are identical or different, are chosen from divalent organic groups which are bonded to the adjacent silicon atom via a carbon-silicon bond and/or to the polyoxyalkylene (CₙH₂ₙO) block via an oxygen atom,

the average molecular weight of each siloxane block ranges from 400 to 10,000 a.m.u., that of each polyoxyalkylene block ranging from 300 to 10,000 a.m.u.,

the siloxane blocks represent from 10% to 95% by weight of the block copolymer,

it being possible for the number-average molecular weight of the block copolymer to range from 2500 to 1,000,000, for instance, between 3,000 and 200,000 and, further for example, between 6,000 and 100,000.

In at least one embodiment of the present disclosure, R and R′ are chosen from linear or branched alkyl radicals, such as, for example, methyl, ethyl, propyl, butyl, pentyl, hexyl, octyl, decyl, or dodecyl radicals; aryl radicals, such as, for example, phenyl or naphthyl radicals; aralkyl radicals, such as, for example, benzyl or phenylethyl radicals; and aralkyl or alkylaryl radicals, such as, for example tolyl or xylyl radicals.

In at least one embodiment, Z, which are identical or different, are chosen from the following groups: —R″—, —R″—CO—, —R″—NHCO—, —R″—NH—CO—NH—R′″—, and —R″—OCONH—R′″—NHCO—; R″ being a linear or branched, divalent, C₁-C₆ alkylene group, for example, linear or branched ethylene, propylene or butylene, and R′″ being a divalent alkylene group or a divalent arylene group, such as —C₆H₄—, —C₆H₄—C₆H₄—, —C₆H₄—CH₂—C₆H₄—, or —C₆H₄—C(CH₃)₂C₆H₄—.

In at least one further embodiment of the present disclosure, Z, which are identical or different, are chosen from divalent alkylene radicals, for instance —C₃H₆— radicals or C₄H₈ radicals, which are linear or branched.

The preparation of the block copolymers is described, for example, in European Patent Application No. EP-A-0 492 657.

Such products are, for example, sold under the name Silicone Fluid FZ-2172 by OSI.

The silicones used can be provided in the form of aqueous solutions, that is to say dissolved, or optionally in the form of aqueous dispersions or microdispersions or of aqueous emulsions.

The silicone(s) which can be used in the cosmetic composition can also be silicone gum(s).

The silicone gums which can be used in the cosmetic composition are, for example, polydiorganosiloxanes having high weight-average molecular weights ranging from 200,000 to 1,000,000 a.m.u., used alone or as a mixture in a solvent. This solvent can be chosen from volatile silicones, polydimethylsiloxane (PDMS) oils, polyphenylmethylsiloxane (PPMS) oils, isoparaffins, polyisobutylenes, methylene chloride, pentane, dodecane, tridecanes and mixtures thereof.

Non-limiting mention may be made of the following products:

polydimethylsiloxane,

polydimethylsiloxane/methylvinylsiloxane gums,

polydimethylsiloxane/diphenylmethylsiloxane,

polydimethylsiloxane/phenylmethylsiloxane,

polydimethylsiloxane/diphenylsiloxane/methylvinylsiloxane.

Non-limiting examples of products which can be used in accordance with the disclosure are mixtures, such as:

mixtures formed from a polydimethylsiloxane hydroxylated at the chain end (named dimethiconol according to the nomenclature of the CTFA dictionary) and from a cyclic polydimethylsiloxane (named cyclomethicone according to the nomenclature of the CTFA dictionary), such as the product Q2 1401 sold by Dow Corning;

mixtures formed from a polydimethylsiloxane gum with a cyclic silicone, such as the product SF 1214 Silicone Fluid from General Electric; this product is an SE 30 gum corresponding to a dimethicone, having a molecular weight of 500,000 a.m.u., dissolved in the SF 1202 Silicone Fluid oil corresponding to decamethylcyclopentasiloxane;

mixtures of two PDMSs with different viscosities and, for instance, of a PDMS gum and of a PDMS oil, such as the product SF 1236 from General Electric. The product SF 1236 is the mixture of an SE 30 gum defined above, with a viscosity of 20 m²/s, and of an SF 96 oil with a viscosity of 5.10⁻⁶ m²/s. This product can, for example, comprise 15% by weight of SE 30 gum and 85% by weight of an SF 96 oil.

The silicone or silicones which can be used in the cosmetic composition can also be, for instance, aminated silicones.

As used herein, the term “aminated silicone” is understood to mean any silicone comprising at least one primary, secondary or tertiary amine functional group or one quaternary ammonium group.

The aminated silicones used in the cosmetic composition, as disclosed herein, can be chosen from:

(a) the compounds corresponding to the following formula (XI):
(R¹)ₐ(T)₍₃₋ₐ₎-Si[OSi(T)₂]ₙ-[OSi(T)_{b}(R¹)_{(2-b)}]ₘ—OSi(T)₍₃₋ₐ₎-(R¹)ₐ (XI)
wherein:

T, which are identical or different, are chosen from hydrogen and from phenyl, hydroxyl (—OH), C₁-C₈ alkyl, such as methyl, and C₁-C₈ alkoxy, such as methoxy radicals,

a is a number ranging from 0 to 3, and, in at least one embodiment, 0,

b is chosen from 0 and 1, and, in at least one embodiment, 1,

m and n are numbers such that the sum (n+m) can vary, for example, from 1 to 2,000 and, such as, between 50 and 150, it being possible for n to equal a number from 0 to 1,999 and, such as, between 49 and 149 and it being possible for m to be a number from 1 to 2000 and, such as, between 1 and 10;

R¹ is a monovalent radical of formula —C_{q}H_{2q}L wherein q is an integer ranging from 2 to 8 and L is an aminated group, optionally quaternized, chosen from the groups:

—N(R²)—CH₂—CH₂—N(R²)₂;

—N(R²)₂;

—N⁺(R²)₃Q⁻;

—N⁺(R²)(H)₂Q⁻;

—N⁺(R²)₂ HQ⁻; and

—N(R²)—CH₂—CH₂—N⁺(R²)(H)₂Q⁻,

wherein R² is chosen from hydrogen, and from phenyl, benzyl and saturated monovalent hydrocarbon radicals, for example a C₁-C₂₀ alkyl radical, and Q⁻ is chosen from halide ions, such as, fluoride, chloride, bromide, or iodide.

As disclosed herein, the aminated silicones corresponding to the definition of the formula (XI) are, for instance, chosen from the compounds corresponding to the following formula (XII):

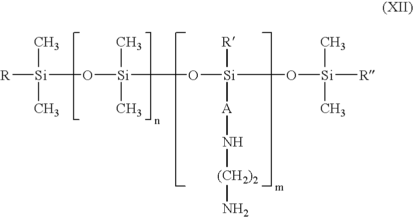

wherein:

R, R′ and R″, which are identical or Different, are Chosen from C₁-C₄ Alkyl radicals, such as CH₃, C₁-C₄ alkoxy radicals, such as methoxy or OH;

A is chosen from linear or branched C₃-C₈, for instance C₃-C₆, alkylene radicals;

m and n are integers depending on the molecular weight, the sum of which ranges from 1 to 2,000.

According to one embodiment of the present disclosure, R, R′ and R″, which are identical or different, are chosen from C₁-C₄ alkyl radicals and hydroxyl radicals, A is chosen from C₃ alkylene radicals and m and n are such that the weight-average molecular weight of the compound ranges from 5000 to 500,000 a.m.u. The compounds of this type are named “amodimethicone” in the CTFA dictionary.

According to another embodiment of the present disclosure, R, R′, and R″, which are identical or different, are chosen from C₁-C₄ alkoxy radicals and hydroxyl radicals, at least one of the R or R″ radicals is an alkoxy radical and A is chosen from C₃ alkylene radicals. In at least one embodiment, the hydroxyl/alkoxy molar ratio ranges, for example, from 0.2/1 to 0.4/1, and, in a further embodiment, is equal to 0.3/1. Furthermore, m and n are such that the weight-average molecular weight of the compound ranges from 2,000 to 10⁶ a.m.u. In one embodiment, n ranges from 0 to 999 and m ranges from 1 to 1,000, the sum of n and m ranging from 1 to 1,000.

Non-limiting examples include, in this category of compounds, inter alia, the product Belsil®ADM 652 sold by Wacker.

According to yet another embodiment of the present disclosure, R and R″, which are different, are chosen from C₁-C₄ alkoxy radicals and hydroxyl radicals, at least one of the R or R″ radicals is an alkoxy radical, R′ is chosen from methyl radicals and A is chosen from C₃ alkylene radicals. The hydroxyl/alkoxy molar ratio ranges, for example, from 1/0.8 to 1/1.1 and, in one embodiment, is equal to 1/0.95. Furthermore, m and n are such that the weight-average molecular weight of the compound ranges from 2,000 to 200,000 a.m.u. In one embodiment, n ranges from 0 to 999 and m ranges from 1 to 1000, the sum of n and m ranging from 1 to 1,000.

Non-limiting mention may be made of the product Fluid WR® 1300 sold by Wacker.

According to another embodiment of the present disclosure, R and R″ are chosen from hydroxyl radicals, R′ is chosen from methyl radicals and A is chosen from C₄-C₈, for instance C₄, alkylene radicals. Furthermore, m and n are such that the weight-average molecular weight of the compound ranges from 2,000 to 10⁶ a.m.u. In one embodiment, n ranges from 0 to 1,999 and m ranges from 1 to 2,000, the sum of n and m ranging from 1 to 2000.

A product of this type is sold, for example, under the name DC 28299 by Dow Corning.

It should be noted that the molecular weight of these silicones is determined by gel permeation chromatography (ambient temperature, polystyrene standard; styragem μ columns; eluent THF; flow rate of 1 mm/m; 200 μl of a 0.5% by weight solution of silicone in THF are injected and the technique is carried out by refractometry and UV spectrometry).

A product corresponding to the definition of the formula (XI) is, for example, the polymer named “trimethylsilylamodimethicone” in the CTFA dictionary, corresponding to the following formula (XIII):

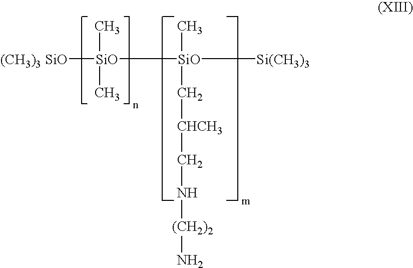

wherein n and m have the meanings given above in accordance with the formula (XI).

Such compounds are described, for example, in European Patent Application No. EP-A-0 095 238; a compound of formula (XIII) is, for example, sold under the name Q2-8220 by OSI.

(b) the compounds corresponding to the following formula (XIV): 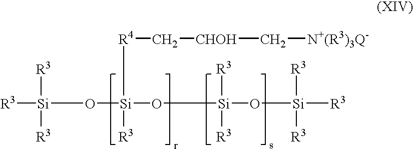
wherein:

R³, which are identical or different, are chosen from monovalent C₁-C₁₈ hydrocarbon radicals and, in one embodiment are chosen from C₁-C₁₈ alkyl radicals and C₂-C₁₈ alkenyl radicals, and in a further embodiment are methyl radicals;

R⁴ is chosen from divalent hydrocarbon radicals, such as C₁-C₁₈ alkylene radicals and divalent C₁-C₁₈, for example C₁-C₈, alkyleneoxy radicals;

Q⁻ is chosen from halide ions, such as chloride;

r represents a mean statistical value ranging from 2 to 20, such as between 2 and 8;

s represents a mean statistical value of 20 to 200, such as between 20 and 50.

Such compounds are described, for example, in U.S. Pat. No. 4,185,087.

A compound within this category is that sold by Union Carbide under the name “Ucar Silicone ALE 56”.

c) the quaternary ammonium silicones of formula (XV): 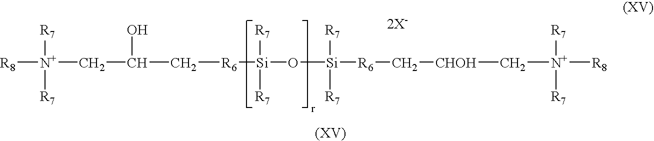
wherein:

R₇, which are identical or different, are chosen from monovalent hydrocarbon radicals comprising 1 to 18 carbon atoms and, in one embodiment are chosen from C₁-C₁₈ alkyl radicals, C₂-C₁₈ alkenyl radicals or rings comprising 5 or 6 carbon atoms, for example methyl;

R₆ is chosen from divalent hydrocarbon radicals, such as C₁-C₁₈ alkylene radicals and divalent C₁-C₁₈, for example C₁-C₈, alkyleneoxy radicals connected to the Si via an Si—C bond;

R₈, which are identical or different, are chosen from hydrogen, monovalent hydrocarbon radicals comprising 1 to 18 carbon atoms, for instance C₁-C₁₈ alkyl radicals or C₂-C₁₈ alkenyl radicals, and —R₆—NHCOR₇ radicals;

X⁻ is chosen from anions, such as halide ions, for instance chloride, or organic acid anions (acetate, and the like);

r represents a mean statistical value ranging from 2 to 200, such as between 5 and 100.

These silicones are, for example, described in European Patent Application No. EP-A-0 530 974.

d) the aminated silicones of formula (XVI): 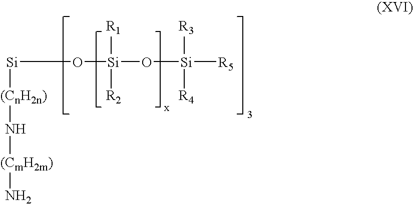
wherein:

R₁, R₂, R₃ and R₄, which are identical or different, are chosen from C₁-C₄ alkyl radicals and phenyl groups,

R₅ is chosen from C₁-C₄ alkyl radicals and hydroxyl groups,

n is an integer ranging from 1 to 5,

m is an integer ranging from 1 to 5,

and wherein x is chosen so that the amine number ranges from 0.01 to 1 meq/g.

The silicones which are, for instance, are the polysiloxanes comprising aminated groups, such as amodimethicones or trimethylsilylamodimethicones (CTFA, 4^{th} edition, 1997), and, further for example, silicones comprising quaternary ammonium groups.

When these compounds are employed, for example, one embodiment is their joint use with cationic and/or non-ionic surface-active agents.

By way of example, the product sold under the name “Cationic Emulsion DC 929” by Dow Corning can be used, which comprises, in addition to the amodimethicone, a cationic surface-active agent comprising a mixture of products corresponding to the formula:

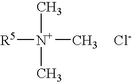

wherein:

R⁵ is chosen from C₁₄-C₂₂ alkenyl and/or alkyl radicals derived from tallow fatty acids, and known under the CTFA name “tallowtrimonium chloride”, in combination with a non-ionic surface-active agent of formula:

C₉H₁₉—C₆H₄—(OC₂H₄)₁₀—OH, known under the CTFA name “Nonoxynol 10”.

Non-limiting mention of the product sold under the name “Cationic Emulsion DC 939” by Dow Corning can be made, which comprises, in addition to the amodimethicone, a cationic surface-active agent which is trimethylcetylammonium chloride and a non-ionic surface-active agent of formula: C₁₃H₂₇—(OC₂H₄)₁₂—OH, known under the CTFA name “trideceth-12”.

Another commercial product which can be used according to the disclosure is the product sold under the name “Dow Corning Q2 7224” by Dow Corning, comprising, in combination, the trimethylsilylamodimethicone of formula (XVI) described above, a non-ionic surface-active agent of formula: C₈H₁₇—C₆H₄—(OCH₂CH₂)₄₀—OH, known under the CTFA name “octoxynol-40”, a second non-ionic surface-active agent of formula: C₁₂H₂₅—(OCH₂—CH₂)₆—OH, known under the CTFA name “isolaureth-6”, and propylene glycol.

The silicone(s) is (or are) present in the composition in an amount ranging from 0.1 to 30% by weight, for example between 0.2 and 20% by weight and, further for example, between 0.5 and 10% by weight, with respect to the total weight of the composition.

In one embodiment of the present disclosure, the cosmetic composition disclosed herein can additionally comprise a non-silicone fatty substance, such as vegetable, animal, mineral and synthetic oils, fatty alcohols, fatty acids, and waxes.

The substance(s) is (or are) present in the composition in an amount ranging from 0.1 to 30% by weight, for instance between 0.2 and 20% by weight and, further for example, between 0.5 and 10% by weight, with respect to the total weight of the composition.

As used herein, the term “fatty alcohol” is understood to mean any pure, saturated or unsaturated, linear or branched, fatty alcohol comprising at least 8 carbon atoms. The Fatty alcohol can be oxyalkylenated or glycerolated.

The fatty alcohol can exhibit the structure R—OH, wherein R is chosen from saturated or unsaturated and linear or branched radicals comprising 8 to 40 carbon atoms and, for example, between 8 and 30 carbon atoms; further for example, R is chosen from C₁₂-C₂₄ alkyl and C₁₂-C₂₄ alkenyl groups. R can be substituted by at least one hydroxyl group.

Non-limiting mention may be made, as example of fatty alcohols, of lauryl, cetyl, dodecyl, decyl, stearyl, oleyl, behenyl, linoleyl, undecylenyl, palmitoleyl, arachidonyl and erucyl alcohols, and their mixtures.

The fatty alcohol can represent a mixture of fatty alcohols, which means that several types of fatty alcohols can coexist in a commercial product in the form of a mixture.

Non-limiting mention may be made, as a mixture of fatty alcohols, of cetylstearyl or cetearyl alcohol.

In at least one embodiment of the present disclosure, the nonoxyalkylenated fatty alcohol is solid or pasty at a temperature of 25° C. As used herein, the term “fatty alcohol which is solid or pasty at 25° C.” is understood to mean a fatty alcohol exhibiting a viscosity, measured with a rheometer with a shear rate of 1 s⁻¹, of greater than or equal to 1 Pa·s.

The fatty alcohols used in the cosmetic composition according to the disclosure can be, for instance, cetyl alcohol and cetearyl alcohol.

As used herein, the term “fatty acids” is understood to mean any pure, saturated or unsaturated, linear or branched, carboxylic acid comprising least 8 carbon atoms. Non limiting examples of fatty acids include lauric acid and oleic acid.

In another embodiment of the present disclosure, the cosmetic composition can additionally comprise at least one additional fixing polymer.

As used herein, the term “fixing polymer” is understood to mean any polymer which makes it possible to confer a form or to retain a given form or hairstyle.

The cosmetic composition can additionally comprise at least one additional fixing polymer other than the polymers described above.

The additional fixing polymers which can be used in the cosmetic composition according to the disclosure are chosen, for example, from cationic, anionic, amphoteric, and non-ionic polymers and their mixtures other than the fixing material described above.

As used herein, the term “cationic polymer” is understood to mean any polymer comprising cationic groups and/or groups which can be ionized to give cationic groups.

The cationic fixing polymers which can be used in the cosmetic composition according to the disclosure can be chosen, for example, from polymers comprising primary, secondary, tertiary, and/or quaternary amine groups, which form part of the polymer chain or are directly connected to the latter, and having a number-average molecular weight ranging from 500 to 5,000,000, for instance, from between 1,000 and 3,000,000.

Non-limiting mention may be made, among these polymers, of the following cationic polymers:

(1) homopolymers or copolymers of acrylic or methacrylic esters or amides, possessing amino functional groups, comprising at least one of the units of the following formulae: 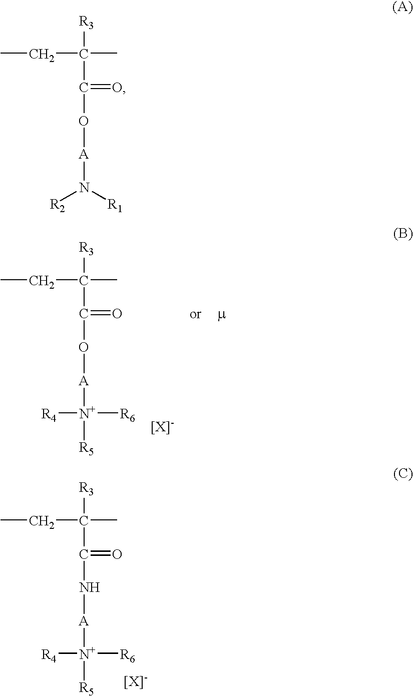
wherein:

R₁ and R₂, which are identical or different, are chosen from hydrogen and alkyl groups comprising 1 to 6 carbon atoms;

R₃ is chosen from hydrogen and CH₃ groups;

A is chosen from linear or branched alkyl groups comprising 1 to 6 carbon atoms and from hydroxyalkyl groups comprising 1 to 4 carbon atoms;

R₄, R₅ and R₆, which are identical or different, are chosen from alkyl groups comprising 1 to 18 carbon atoms and from benzyl groups;

[X]⁻ is chosen from methosulphate anions and halides, such as chloride or bromide.

The copolymers of family (1) can additionally comprise at least one unit derived from comonomers which can be chosen from the family of the acrylamides, methacrylamides, diacetone acrylamides, acrylamides, and methacrylamides substituted on the nitrogen by lower (C₁-C₄)alkyl groups, groups derived from acrylic or methacrylic acids or from their esters, vinyllactams, such as vinylpyrrolidone or vinylcaprolactam, or vinyl esters.

Thus, non-limiting mention may be made, among these copolymers of family (1), of:

copolymers of acrylamide and of dimethylaminoethyl methacrylate which are quaternized with dimethyl sulphate or with a methyl halide, such as that sold under the name Hercofloc® by Hercules,

copolymers of acrylamide and of methacryloyloxyethyltrimethylammonium chloride, described, for example, in European Patent Application No. EP-A-0 080 976 and sold under the name Binaquat P 100 by Ciba-Geigy,

copolymers of acrylamide and of methacryloyloxyethyltrimethylammonium methosulphate, such as that sold under the name Reten by Hercules,

vinylpyrrolidone/dialkylaminoalkyl acrylate or methacrylate copolymers, which may or may not be quaternized, such as the products sold under the name “Gafquat®” by ISP, such as, for example, “Gafquat® 734” or “Gafquat® 755”, or the products named “Copolymer® 845, 958 and 937”. These polymers are described in detail, for example, in French Patent Nos. 2 077 143 and 2 393 573,

dimethylaminoethylmethacrylate/vinylcaprolactam/vinyl-pyrrolidone terpolymers, such as the product sold under the name Gaffix® VC 713 by ISP, and

vinylpyrrolidone/quaternized dimethylaminopropylmethacrylamide copolymers, such as, for example, the product sold under the name “Gafquat® HS 100” by ISP.

(2) Cationic polysaccharides, for example comprising quaternary ammonium, such as those described in U.S. Pat. Nos. 3,589,578 and 4,031,307, such as guar gums comprising trialkylammonium cationic groups. Such products are sold, for instance, under the trade names Jaguar C13 S, Jaguar C15, and Jaguar C17 by Meyhall.

(3) Quaternary copolymers of vinylpyrrolidone and of vinylimidazole.

(4) Chitosans or their salts; the salts which can be used are, for instance, chitosan acetate, lactate, glutamate, gluconate or pyrrolidonecarboxylate.

Non-limiting mention may be made, among these compounds, of the chitosan having a degree of deacetylation of 90.5% by weight sold under the name Kytan Brut Standard by Aber Technologies or the chitosan pyrrolidonecarboxylate sold under the name Kytamer® PC by Amerchol.

(5) Cationic cellulose derivatives, such as the copolymers of cellulose or of cellulose derivatives grafted with a water-soluble monomer comprising a quaternary ammonium and described, for instance, in U.S. Pat. No. 4,131,576, such as hydroxyalkylcelluloses, for example hydroxymethyl-, hydroxyethyl-, or hydroxypropylcelluloses, grafted, for instance, with a methacryloyloxyethyltrimethylammonium, methacrylamidopropyltrimethylammonium, or dimethyldiallylammonium salt.

The marketed products corresponding to this definition can be, for example, the products sold under the names “Celquat L 200” and “Celquat H 100” by National Starch.

The anionic fixing polymers generally used are polymers comprising groups derived from carboxylic, sulphonic or phosphoric acid and have a number-average molecular weight ranging from 500 to 5,000,000.

The carboxyl groups are contributed by unsaturated mono- or dicarboxylic acid monomers such as those corresponding to the formula: 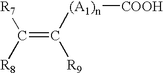
wherein:

n is an integer ranging from 0 to 10,

A₁ is chosen from methylene groups, optionally connected to the carbon atom of the unsaturated group or to the neighboring methylene group when n is greater than 1 via a heteroatom, such as oxygen or sulphur,

R₇ is chosen from hydrogen and phenyl or benzyl groups,

R₈ is chosen from hydrogen, lower alkyl groups, and carboxyl groups,

R₉ is chosen from hydrogen, lower alkyl groups, and —CH₂—COOH, phenyl, or benzyl groups.

In the abovementioned formula, a lower alkyl group can be chosen from groups comprising 1 to 4 carbon atoms and, for example, methyl and ethyl groups.

Non-limiting examples of the anionic fixing polymers comprising carboxyl groups according to the disclosure include:

A) Homo- or copolymers of acrylic or methacrylic acid or their salts and, for example, the products sold under the names Versicol® E or K by Allied Colloid and Ultrahold® by BASF, the copolymers of acrylic acid and of acrylamide sold in the form of their sodium salts under the names Reten 421, 423 or 425 by Hercules or the sodium salts of polyhydroxycarboxylic acids.

B) Copolymers of acrylic or methacrylic acid with a monoethylenic monomer, such as ethylene, styrene, vinyl esters or esters of acrylic or methacrylic acid, optionally grafted onto a polyalkylene glycol, such as polyethylene glycol, and optionally crosslinked. Such polymers are described, for example, in French Patent No. 1 222 944 and German Application No. 2 330 956, the copolymers of this type comprising, in their chain, an optionally N-alkylated and/or hydroxyalkylated acrylamide unit, such as described, for example, in Luxembourgian Patent Applications Nos. 75370 and 75371 or provided under the name Quadramer by American Cyanamid. Non-limiting mention may also be made of methacrylic acid/ethyl acrylate/tert-butyl acrylate terpolymers, such as the product sold under the name Luvimer® 100 P by BASF.

Non-limiting mention may also be made of the methacrylic acid/acrylic acid/ethyl acrylate/methyl methacrylate copolymers in aqueous dispersion sold under the name Amerhold® DR 25 by Amerchol.

C) Copolymers of crotonic acid, such as those comprising, in their chain, vinyl acetate or propionate units and optionally other monomers, such as allyl or methallyl esters, vinyl ether or vinyl ester of a linear or branched saturated carboxylic acid comprising a long hydrocarbon chain, such as those comprising at least 5 carbon atoms, it optionally being possible for these polymers to be grafted or crosslinked, or alternatively another monomer which is a vinyl, allyl or methallyl ester of an α- or β-cyclic carboxylic acid. Such polymers are described, inter alia, in French Patents Nos. 1 222 944, 1 580 545, 2 265 782, 2 265 781, 1 564 110 and 2 439 798. Commercial products coming within this class are the Resins 28-29-30, 26-13-14 and 28-13-10 sold by National Starch.

D) Copolymers of C₄-C₈ monounsaturated carboxylic acids or anhydrides chosen from:

copolymers comprising (i) at least one maleic, fumaric or itaconic acids or anhydrides and (ii) at least one monomer chosen from vinyl esters, vinyl ethers, vinyl halides, phenylvinyl derivatives, or acrylic acid and its esters, the anhydride functional groups of these copolymers optionally being monoesterified or monoamidated. Such polymers are described, for instance, in U.S. Pat. Nos. 2,047,398, 2,723,248 and 2,102,113 and GB Patent No. 839 805. Commercial products are, for example, those sold under the names Gantrez® AN or ES by ISP;

copolymers comprising (i) at least one maleic, citraconic or itaconic anhydride units and (ii) at least one monomer chosen from allyl and methallyl esters, optionally comprising at least one acrylamide, methacrylamide, α-olefin, acrylic or methacrylic ester, acrylic or methacrylic acid, or vinylpyrrolidone groups in their chain, the anhydride functional groups of these copolymers optionally being monoesterified or monoamidated.

These polymers are, for example, described in French Patent Application Nos. FR-A-2 350 384 and FR-A-2 357 241 of L'Oreal.

E) Polyacrylamides comprising carboxylate groups.

The homopolymers and copolymers comprising sulpho groups are polymers comprising vinylsulphonic, styrenesulphonic, naphthalenesulphonic, or acrylamidoalkylsulphonic units.

These polymers can, for example, be chosen from:

salts of polyvinylsulphonic acid having a molecular weight ranging from 1,000 to 100,000 a.m.u., as well as copolymers with an unsaturated comonomer, such as acrylic or methacrylic acids and their esters, as well as acrylamide or its derivatives, vinyl ethers, and vinylpyrrolidone;

salts of polystyrenesulphonic acid, such as the sodium salts sold, for example, under the names Flexan® 500 and Flexan® 130 by National Starch. These compounds are described in French Patent 2 198 719;

salts of polyacrylamidosulphonic acids, such as those mentioned, for example, in U.S. Pat. No. 4,128,631 and, for instance, the polyacrylamidoethylpropanesulphonic acid sold under the name Cosmedia Polymer HSP 1180 by Henkel.

For instance, the anionic fixing polymers are chosen from acrylic acid copolymers, such as the acrylic acid/ethyl acrylate/N-tert-butylacrylamide terpolymers sold, for example, under the name Ultrahold® Strong by BASF, copolymers derived from crotonic acid, such as the vinyl acetate/vinyl tert-butylbenzoate/crotonic acid terpolymers and the crotonic acid/vinyl acetate/vinyl neododecanoate terpolymers sold, for example, under the name Resin 28-29-30 by National Starch, polymers derived from maleic, fumaric or itaconic acids or anhydrides with vinyl esters, vinyl ethers, vinyl halides, phenylvinyl derivatives, or acrylic acid and its esters, such as the monoesterified methyl vinyl ether/maleic anhydride copolymers sold, for example, under the name Gantrez® by ISP, the copolymers of methacrylic acid and of methyl methacrylate sold under the name Eudragit® L by Rohm Pharma, the copolymers of methacrylic acid and of ethyl acrylate sold under the name Luvimer® MAEX or MAE by BASF, the vinyl acetate/crotonic acid copolymers sold, for example, under the name Luviset CA 66 by BASF and the vinyl acetate/crotonic acid copolymers grafted by polyethylene glycol sold under the name Aristoflex® A by BASF.

In at least one embodiment, the anionic fixing polymers that can be used herein are chosen from the monoesterified methyl vinyl ether/maleic anhydride copolymers sold under the name Gantrez® ES 425 by ISP, the acrylic acid/ethyl acrylate/N-tert-butylacrylamide terpolymers sold under the name Ultrahold® Strong by BASF, the copolymers of methacrylic acid and of methyl methacrylate sold under the name Eudragit® L by Rohm Pharma, the vinyl acetate/vinyl tert-butylbenzoate/crotonic acid terpolymers and the crotonic acid/vinyl acetate/vinyl neododecanoate terpolymers sold under the name Resin 28-29-30 by National Starch or the copolymers of methacrylic acid and of ethyl acrylate sold under the name Luvimer® MAEX or MAE by BASF.

The amphoteric fixing polymers which can be used in accordance with the disclosure can be chosen from polymers comprising B and C units distributed randomly in the polymer chain, where B is chosen from units deriving from monomers comprising at least one basic nitrogen atom and C is chosen from units comprising acidic monomers comprising at least one carboxyl or sulpho group or else B and C are chosen from groups comprising zwitterionic carboxybetaine or sulphobetaine monomers;

In another embodiment, B and C are chosen from cationic polymer chains comprising primary, secondary, tertiary, and quaternary amine groups, wherein at least one of the amine groups carries a carboxyl or sulpho group connected via a hydrocarbon group, or else B and C form part of a chain of a polymer comprising an α,β-dicarboxyethylene unit, one of the carboxyl groups of which has been reacted with a polyamine comprising at least one primary or secondary amine groups.

Non-limiting examples of the amphoteric fixing polymers, as disclosed herein, are chosen from the following polymers:

1) Copolymers comprising acidic vinyl units and comprising basic vinyl units, such as those resulting from the copolymerization of a monomer derived from a vinyl compound carrying a carboxyl group, such as acrylic acid, methacrylic acid, maleic acid, or α-chloroacrylic acid, and of a basic monomer derived from a substituted vinyl compound comprising at least one basic atom, such as dialkylaminoalkyl methacrylate and acrylate or dialkylaminoalkylmethacrylamide and -acrylamide. Such compounds are described in, for example, U.S. Pat. No. 3,836,537.

2) Polymers comprising units derived:

a) from at least one monomer chosen from acrylamides and methacrylamides substituted on the nitrogen atom by an alkyl group,

b) from at least one acidic comonomer comprising at least one reactive carboxyl group, and

c) from at least one basic comonomer, such as esters comprising primary, secondary, tertiary, and quaternary amine substituents of acrylic and methacrylic acids and the quaternization product of dimethylaminoethyl methacrylate with dimethyl or diethyl sulphate.

Among the N-substituted acrylamides or methacrylamides according to the present disclosure, non-limiting mention may be made of the compounds wherein the alkyl groups comprise from 2 to 12 carbon atoms, for example, N-ethylacrylamide, N-tert-butylacrylamide, N-tert-octylacrylamide, N-octylacrylamide, N-decylacrylamide or N-dodecylacrylamide, and the corresponding methacrylamides.

The acidic comonomers are, for example, chosen from acrylic, methacrylic, crotonic, itaconic, maleic and fumaric acids, and alkyl monoesters comprising 1 to 4 carbon atoms of maleic or fumaric acids or anhydrides.

Non-limiting examples of basic comonomers include aminoethyl, butylaminoethyl, N,N′-dimethylaminoethyl, and N-tert-butylaminoethyl methacrylates.

Additional non-limiting examples the copolymers can be used for which the CTFA name (4th Ed., 1991) is Octylacrylamide/acrylates/butylaminoethyl methacrylate copolymer, such as the products sold under the name Amphomer® or Lovocryl® 47 by National Starch.

(3) Partially or completely acylated and crosslinked polyaminoamides deriving from polyaminoamides of following formula (XVII): [symbol]
CO—R₁₀—CO-Z [symbol] (XVII)

wherein:

R₁₀ is chosen from divalent groups derived from a saturated dicarboxylic acid, from an aliphatic mono- or dicarboxylic acid comprising an ethylenic double bond, from an ester of a lower alkanol comprising 1 to 6 carbon atoms of these acids, and from a group derived from the addition of any one of the said acids with a bisprimary or bissecondary amine, and

Z is chosen from groups deriving from a bisprimary, mono- and bissecondary polyalkylenepolyamine and, for instance, comprising the following units:

a) in proportions from 60 to 100 mol %, the group of formula (XVIII):
—NH [symbol] (CH₂)ₓ—NH [symbol] ₚ (XVIII)

where x is equal to 2 and p is equal to 2 or 3, or else x is equal to 3 and p is equal to 2;

this group derived from diethylenetriamine, triethylenetetraamine or dipropylenetriamine;

b) in proportions from 0 to 40 mol %, the above group (XVIII), wherein x is equal to 2 and p is equal to 1 and which derives from ethylenediamine, or the group derived from piperazine:

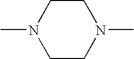

c) in proportions from 0 to 20 mol %, the group —NH—(CH₂)₆—NH— derived from hexamethylenediamine,

these polyaminoamides being crosslinked by addition reaction of a bifunctional crosslinking agent chosen from epihalohydrins, diepoxides, dianhydrides, and bisunsaturated derivatives, by means of 0.025 to 0.35 mol of crosslinking agent per amine group of the polyaminoamide, and acylated by reaction with acrylic acid, chloroacetic acid or an alkanesultone or their salts.

The saturated carboxylic acids can be chosen from acids comprising 6 to 10 carbon atoms, such as adipic, 2,2,4-trimethyladipic, 2,4,4-trimethyladipic, or terephthalic acids, and the acids comprising an ethylenic double bond, such as, for example, acrylic, methacrylic, or itaconic acids.

The alkanesultones used in the acylation can be, for instance, propane- or butanesultone and the salts of the acylating agents can be, for example, the sodium or potassium salts.

(4) Polymers comprising zwitterionic units of formula: 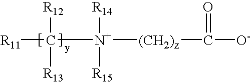
wherein:

R₁₁ is chosen from polymerizable unsaturated groups, such as acrylate, methacrylate, acrylamide, or methacrylamide groups,

y and z are integers ranging from 1 to 3,

R₁₂ and R₁₃, which are identical or different, are chosen from hydrogen and methyl, ethyl, and propyl groups,

R₁₄ and R₁₅, which are identical or different, are chosen from hydrogen and alkyl groups such that the sum of the carbon atoms in R₁₄ and R₁₅ does not exceed 10.

The polymers comprising such units can also comprise units derived from non-zwitterionic monomers, such as dimethyl- or diethylaminoethyl acrylate or methacrylate or alkyl acrylates or methacrylates, acrylamides or methacrylamides, or vinyl acetate.

Non-limiting mention may be made, by way of example, of methyl methacrylate/dimethylcarboxymethylammonioethyl methacrylate copolymers, such as the product sold under the name Diaformer Z301 by Sandoz.

(5) Polymers derived from chitosan comprising monomer units corresponding to the following formulae, and the salts formed by these compounds with bases or acids:

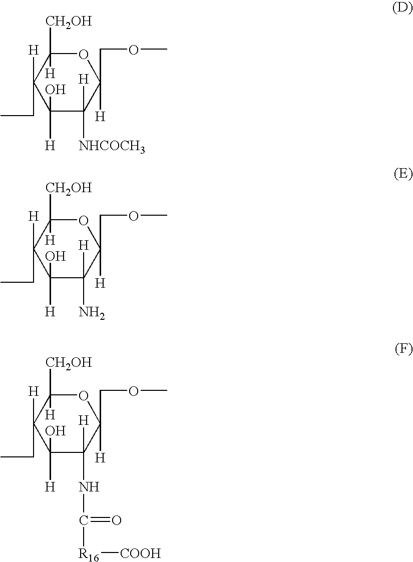

the unit (D) being present in proportions from 0 to 30 mol %, the unit (E) in proportions from 5 to 50 mol % and the unit (F) in proportions from 30 to 90 mol %, it being understood that, in this unit (F), R₁₆ is chosen from groups of formula: 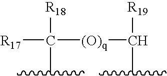
wherein:

if q=0, R₁₇, R₁₈ and R₁₉, which are identical or different, are each chosen from hydrogen, and from methyl, hydroxyl, acetoxy and amino residues, monoalkylamino residues and dialkylamino residues, optionally interrupted by at least one nitrogen atom and/or optionally substituted by at least one amino, hydroxyl, carboxyl, alkylthio or sulpho group, and alkylthio residues wherein the alkyl group carries an amino residue, at least one of the R₁₇, R₁₈ and R₁₉ groups being, in this case, a hydrogen atom;

or, if q=1, R₁₇, R₁₈ and R₁₉ are chosen from hydrogen.

(6) Polymers comprising units corresponding to the formula (XIX), for example, described in French Patent No. FR-A-1 400 366:

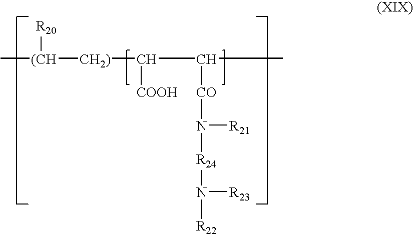

wherein:

R₂₀ is chosen from hydrogen, CH₃O, CH₃CH₂O, and phenyl groups,

R₂₁ is chosen from hydrogen, and lower alkyl groups, such as methyl or ethyl,

R₂₂ is chosen from hydrogen and lower C₁-C₆ alkyl groups, such as methyl or ethyl,

R₂₃ is chosen from lower C₁-C₆ alkyl groups, such as methyl or ethyl, and groups corresponding to the formula: —R₂₄—N(R₂₂)₂, R₂₄ is chosen from —CH₂—CH₂—, —CH₂—CH₂—CH₂— and —CH₂—CH(CH₃)— groups and R₂₂ having the meanings mentioned above.

(7) Polymers derived from the N-carboxyalkylation of chitosan, such as the N-(carboxymethyl)chitosan or the N-(carboxybutyl)chitosan sold under the name “Evalsan” by Jan Dekker.

(8) Amphoteric polymers of the -D-X-D-X- type chosen from:

a) polymers obtained by reaction of chloroacetic acid or sodium chloroacetate with compounds comprising at least one unit of formula (XX):
-D-X-D-X-D- (XX)

where D is chosen from piperazine groups:

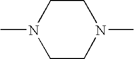

X represents the symbol E or E′; E or E′, which are identical or different, is chosen from bivalent groups which are straight- or branched-chain alkylene groups comprising up to 7 carbon atoms in the main chain which is unsubstituted or substituted by hydroxyl groups and which can additionally comprise oxygen, nitrogen or sulphur atoms or 1 to 3 aromatic and/or heterocyclic rings; the oxygen, nitrogen and sulphur atoms being present in the form of ether, thioether, sulphoxide, sulphone, sulphonium, alkylamine or alkenylamine groups or hydroxyl, benzylamine, amine oxide, quaternary ammonium, amide, imide, alcohol, ester, and/or urethane groups.

b) polymers of formula (XXI):
-D-X-D-X- (XXI)

where D is chosen from piperazine groups:

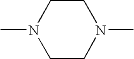

X represents the symbol E or E′ and at least once E′, E having the meaning indicated above and E′ being chosen from bivalent groups which are straight- or branched-chain alkylene groups comprising up to 7 carbon atoms in the main chain which is unsubstituted or substituted by at least one hydroxyl groups and which comprises at least one nitrogen atom, the nitrogen atom being substituted by an alkyl chain optionally interrupted by an oxygen atom and necessarily comprising at least one carboxyl functional groups and at least one hydroxyl functional group and betainized by reaction with chloroacetic acid or sodium chloroacetate.

(9) (C₁-C₅)Alkyl vinyl ether/maleic anhydride copolymers partially modified by semiamidation with an N,N-dialkylaminoalkylamine, such as N,N-dimethylaminopropylamine, or by semiesterification with an N,N-dialkylaminoalkanol. These copolymers can also comprise other vinyl comonomers, such as vinylcaprolactam.

Among the amphoteric fixing polymers described herein, non-limiting mention may be made of those of family (3), such as the copolymers with the CTFA name of Octylacrylamide/acrylates/butylaminoethyl methacrylate copolymer, such as the products sold under the names Amphomer®, Amphomer® LV 71, or Lovocryl® 47 by National Starch, and those of family (4), such as methyl methacrylate/dimethylcarboxymethyl-ammonioethyl methacrylate copolymers, sold, for example, under the name Diaformer® Z301 by Sandoz.

The non-ionic fixing polymers which can be used according to the present disclosure are chosen, for example, from:

polyalkyloxazolines;

vinyl acetate homopolymers;

vinyl acetate copolymers, such as, for example copolymers of vinyl acetate and of acrylic ester, copolymers of vinyl acetate and of ethylene, or copolymers of vinyl acetate and of maleic: ester, for example of dibutyl maleate;

acrylic ester homopolymers and copolymers, such as, for example, copolymers of alkyl acrylates and of alkyl methacrylates, such as the products provided by Rohm & Haas under the names Primal® AC-261 K and Eudragit® NE 30 D, by BASF under the name 8845 or by Hoechst under the name Appretan® N9212;

copolymers of acrylonitrile and of a non-ionic monomer chosen, for example, from butadiene and alkyl(meth)acrylates; non-limiting mention may be made of the products provided under the name CJ 0601 B by Rohm & Haas;

styrene homopolymers;

styrene copolymers, such as, for example, copolymers of styrene and of alkyl (meth)acrylate, such as the products Mowilith® LDM 6911, Mowilith® DM 611 and Mowilith® LDM 6070 provided by Hoechst or the products Rhodopas® SD 215 and Rhodopas® DS 910 provided by Rhodia Chimie, copolymers of styrene, of alkyl methacrylate and of alkyl acrylate, copolymers of styrene and of butadiene or copolymers of styrene, of butadiene and of vinylpyridine;

polyamides;

vinyllactam homopolymers other than vinylpyrrolidone homopolymers, such as the polyvinylcaprolactam sold under the name Luviskol® Plus by BASF; and

vinyllactam copolymers, such as the poly(vinylpyrrolidone/vinyllactam) copolymer sold under the trade name Luvitec® VPC 55K65W by BASF, poly(vinyl-pyrrolidone/vinyl acetate) copolymers, such as those sold under the name PVPVA® S630L by ISP or Luviskol® VA 73, VA 64, VA 55, VA 37 and VA 28 by BASF, and poly(vinylpyrrolidone/vinyl acetate/vinyl propionate) terpolymers, such as, for example, that sold under the name Luviskol® VAP 343 by BASF.

The alkyl groups of the non-ionic polymers mentioned above have, in at least one embodiment, from 1 to 6 carbon atoms.

Non-limiting examples of the fixing polymer include cationic, non-ionic, anionic, or amphoteric polyurethanes which may or may not be functionalized and which may or may not be silicone-comprising, or their mixtures.

Non-limiting examples of the polyurethanes according to the present disclosure include those described in European Patent Application Nos. EP-A-0 751 162, EP-A-0 637 600, EP-A-0 648 485, and French Patent Application No. FR-A-2 743 297, of L'Oreal, and in European Patent Application No. EP-A-0 656 021 and International Patent Application No. WO-A-94/03510 of BASF and European Patent Application No. EP-A-0 619 111 of National Starch.

Additional non-limiting examples of suitable polyurethanes include the products sold under the names Luviset P.U.R.® and Luviset® Si P.U.R. by BASF.

The additional fixing polymer(s) is (or are) present in the cosmetic composition according to the disclosure in an amount ranging from 0.01 to 20% by weight, preferably between 0.05 and 15% by weight and, more preferably between 0.1 and 10% by weight, with respect to the total weight of the cosmetic composition.

The composition according to the disclosure can also comprise additional surfactants.

The ionic surfactant(s) used in the cosmetic composition can be, for instance, cationic surfactant(s).

Non-limiting mention may be made, as examples of cationic surfactants which can be used in the cosmetic composition, of salts of primary, secondary or tertiary fatty amines which are optionally polyoxyalkylenated, quaternary ammonium salts and their mixtures.

Non-limiting mention may be made, as quaternary ammonium salts, of, for example:

those which exhibit the following formula (II):

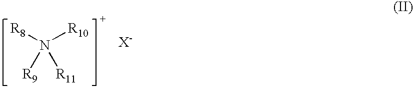

wherein:

the R₈ to R₁₁ radicals, which are identical or different, are chosen from linear or branched aliphatic radicals comprising from 1 to 30 carbon atoms and from aromatic radicals, such as aryl or alkylaryl;

X⁻ is an anion chosen from halides, phosphates, acetates, lactates, (C₂-C₆)alkyl sulphates, alkylsulphonates, and alkylarylsulphonates.

In the formula (II) above, the aliphatic radicals can comprise heteroatoms, such as, oxygen, nitrogen, sulphur, and halogens.

These aliphatic radicals are, for example, chosen from alkyl radicals, alkoxy radicals, polyoxy(C₂-C₆)alkylene radicals, alkylamido radicals, (C₁₂-C₂₂)alkylamido(C₂-C₆)alkyl radicals, (C₁₂-C₂₂)alkyl acetate radicals and hydroxyalkyl radical comprising from 1 to 30 carbon atoms.

imidazoline quaternary ammonium salts, for example, those of following formula (III):

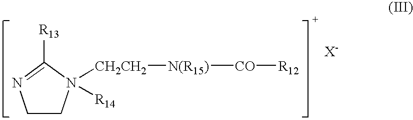

wherein:

R₁₂ is chosen from alkenyl and alkyl radicals comprising 8 to 30 carbon atoms, for example derivatives of tallow fatty acids,

R₁₃ is chosen from hydrogen, C₁-C₄ alkyl radicals, and alkenyl or alkyl radicals comprising 8 to 30 carbon atoms,

R₁₄ is chosen from C₁-C₄ alkyl radicals, R₁₅ is chosen from hydrogen and C₁-C₄ alkyl radicals and X⁻ is an anion chosen from halides, phosphates, acetates, lactates, alkylsulphates, alkylsulphonates, and alkylarylsulphonates.

In one embodiment of the present disclosure, R₁₂ and R₁₃ are chosen from mixtures of alkenyl or alkyl radicals comprising 2 to 21 carbon atoms, for example derivatives of tallow fatty acids, R₁₄ is chosen from methyl radicals R₁₅ is chosen from hydrogen. Such a product is, for example, sold under the name Rewoquat® W 75 by Rewo;

di(quaternary ammonium) salts of formula (IV): 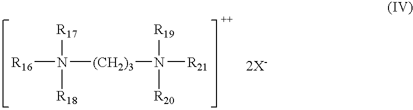
wherein:

R₁₆ is chosen from aliphatic radicals comprising from 16 to 30 carbon atoms,

R₁₇, R₁₈, R₁₉, R₂₀ and R₂₁, which are identical or different, are chosen from hydrogen and an alkyl radicals comprising from 1 to 4 carbon atoms, and

X⁻ is an anion chosen from halides, acetates, phosphates, nitrates, and methylsulphates. Such di(quaternary ammonium) salts comprise, for instance, propanetallowediammonium dichloride;

quaternary ammonium salts comprising at least one ester functional group, such as those of the following formula (V): 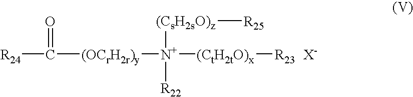
wherein:

R₂₂ is chosen from C₁-C₆ alkyl radicals and C₁-C₆ hydroxyalkyl or dihydroxyalkyl radicals;

R₂₃ is chosen from:

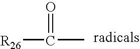

saturated or unsaturated, linear or branched, C₁-C₂₂ hydrocarbon radicals R₂₇,

hydrogen;

R₂₅ is chosen from:

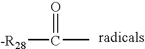

the saturated or unsaturated, linear or branched, C₁-C₆ hydrocarbon radicals R₂₉,

hydrogen;

R₂₄, R₂₆ and R₂₈, which are identical or different, are chosen from saturated or unsaturated, linear or branched, C₇-C₂₁ hydrocarbon radicals;

r, s and t, which are identical or different, are integers ranging from 2 to 6;

y is an integer ranging from 1 to 10;

x and z, which are identical or different, are integers ranging from 0 to 10;

X⁻ is an organic or inorganic, simple or complex anion;

with the proviso that the sum x+y+z has a value from 1 to 15, that, when x has the value 0, then R₂₃ is R₂₇ and that, when z has the value 0, then R₂₅ is R₂₉.

The R₂₂ alkyl radicals can be linear or branched and, in at least one embodiment, are linear.

For instance, R₂₂ is chosen from methyl, ethyl, hydroxyethyl, and dihydroxypropyl radicals, for instance, methyl or ethyl radicals.

The sum x+y+z can have a value, for example, from 1 to 10.

When R₂₃ is R₂₇, it can comprise hydrocarbon radicals, wherein each radical can be long and comprise 12 to 22 carbon atoms or short and comprise 1 to 3 carbon atoms.

When R₂₅ is an R₂₉ hydrocarbon radical, it, for example, has from 1 to 3 carbon atoms.

In one embodiment of the present disclosure, R₂₄, R₂₆ and R₂₈, which are identical or different, are chosen from saturated or unsaturated, linear or branched, C₁₁-C₂₁ hydrocarbon radicals, for example, from saturated or unsaturated, linear or branched, C₁₁-C₂₁ alkyl and alkenyl radicals.

In another embodiment, for instance, x and z, which are identical or different, have the value 0 or 1.

In at least one embodiment, y is equal to 1.

Still, in another embodiment of the present disclosure, r, s and t, which are identical or different, can have the value 2 or 3 and, in at least one embodiment, are equal to 2.

The anion X⁻ can be, for instance, a halide (chloride, bromide or iodide) or an alkylsulphate, for instance, a methylsulphate. However, methanesulphonate, phosphate, nitrate, tosylate, an anion derived from an organic acid, such as acetate or lactate, or any other anion compatible with the ammonium comprising an ester functional group may also be used.

Further, the anion X⁻ can be, for instance, chloride or methyl sulphate.

In one embodiment of the present disclosure the composition disclosed herein comprises the ammonium salts of formula (V) wherein:

R₂₂ is chosen from methyl or ethyl radicals,

x and y are equal to 1;

z is equal to 0 or 1;

r, s, and t are equal to 2;

R₂₃ is chosen from:

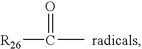

methyl, ethyl, or C₁₄-C₂₂ hydrocarbon radicals, and

hydrogen;

R₂₅ is chosen from:

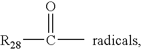

hydrogen;

R₂₄, R₂₆ and R₂₈, which are identical or different, are chosen from saturated or unsaturated, linear or branched, C₁₃-C₁₇ hydrocarbon radicals and, for instance, from saturated or unsaturated, linear or branched, C₁₃-C₁₇ alkyl and alkenyl radicals.

The hydrocarbon radicals, for example, can be linear.

Non-limiting mention may be made, for example, of the compounds of formula (V), such as diacyloxyethyldimethylammonium, diacyloxyethyl(hydroxyethyl)methylammonium, monoacyloxyethyl-(dihydroxyethyl)methylammonium, triacyloxyethylmethylammonium, or monoacyloxyethyl(hydroxyethyl)dimethylammonium salts, such as chloride or methylsulphate, and their mixtures. The acyl radicals can have, for instance, 14 to 18 carbon atoms and, further for instance, can originate from a vegetable oil, such as palm oil or sunflower oil. When the compound comprises several acyl radicals, the latter can be identical or different.

These products are obtained, for example, by direct esterification of triethanolamine, of triisopropanolamine, of alkyldiethanolamine, or of alkyldiisopropanolamine, which are optionally oxyalkylenated, with fatty acids or with mixtures of fatty acids of vegetable or animal origin or by transesterification of their methyl esters. This esterification is followed by a quaternization using an alkylating agent, such as an alkyl (for instance methyl or ethyl) halide, a dialkyl (for instance methyl or ethyl) sulphate, methyl methanesulphonate, methyl para-toluenesulphonate, or glycol or glycerol chlorohydrin.

Such compounds are, for example, sold under the names Dehyquart® by Henkel, Stepanquat® by Stepan, Noxamium® by Ceca or Rewoquat® WE 18 by Rewo-Witco.

The composition according to the disclosure comprises, for instance, a mixture of quaternary ammonium mono-, di- and triester salts, with a majority by weight of diester salts.

As mixture of ammonium salts, non-limiting examples include the mixture comprising 15 to 30% by weight of acyloxyethyl(dihydroxyethyl)methylammonium methyl sulphate, 45 to 60% of diacyloxyethyl(hydroxyethyl)methylammonium methyl sulphate and 15 to 30% of triacyloxyethyl(methyl)ammonium methyl sulphate, the acyl radicals comprising 14 to 18 carbon atoms and originating from optionally partially hydrogenated palm oil.

It is also possible to use the ammonium salts comprising at least one ester functional group described, for example, in U.S. Pat. Nos. 4,874,554 and 4,137,180.

Non-limiting mention may be made among the quaternary ammonium salts of formula (II), on the one hand, to tetraalkylammonium chlorides, such as, dialkyldimethylammonium or alkyltrimethylammonium chlorides, wherein the alkyl radical comprises 12 to 22 carbon atoms, such as behenyltrimethylammonium, distearyldimethylammonium, cetyltrimethylammonium, and benzyldimethylstearylammonium chlorides, or alternatively, on the other hand, to palmitylamidopropyltrimethylammonium chloride or stearamidopropyldimethyl(myristyl acetate)ammonium chloride, sold under the name Ceraphyl® 70 by Van Dyk.

Among the cationic surfactants in the composition of the disclosure, non-limiting mention may be made of quaternary ammonium salts and, for example, cetyltrimethylammonium chloride, behenyltrimethylammonium chloride, and palmitylamidopropyltrimethylammonium chloride.

The ionic surfactants which can be used in the cosmetic composition can also be, for instance, anionic surfactants.

Non-limiting mention may be made, as anionic surfactants which can be used in the cosmetic composition according to the disclosure, of the salts, for example the alkali metal salts, such as the sodium salts, the ammonium salts, the amine salts, the aminoalcohol salts or the alkaline earth metal salts, for example magnesium salts, of the following types: alkyl sulphates, alkyl ether sulphates, alkylamido ether sulphates, alkylaryl polyether sulphates, monoglyceride sulphates; alkylsulphonates, alkylamide sulphonates, alkylaryl sulphonates, α-olefinsulphonates, paraffinsulphonates, alkyl sulphosuccinates, alkyl ether sulphosuccinates, alkylamidesulphosuccinates, alkyl sulphoacetates, acylsarcosinates and acylglutamates, the alkyl and acyl groups of all these compounds comprising 6 to 24 carbon atoms and the aryl group comprising a phenyl or benzyl group.

In one embodiment, C₆-C₂₄ monoalkyl esters of polyglycosidedicarboxylic acids may be used, for instance alkyl glucosidecitrates, alkyl polyglycosidetartrates and alkyl polyglycosidesulphosuccinates, alkyl sulphosuccinamates, acylisethionates and N-acyltaurates, the alkyl or acyl group of all these compounds comprising 12 to 20 carbon atoms.

Another group of anionic surface-active agents which can be used in the compositions of the present disclosure is that of acyl lactylates, the acyl group of which comprises 8 to 20 carbon atoms.

In addition, non-limiting mention may also be made of alkyl-D-galactosideuronic acids and their salts, and also polyoxyalkylenated (C₆-C₂₄)alkyl ether carboxylic acids, polyoxyalkylenated (C₆-C₂₄)alkyl(C₆-C₂₄)aryl ether carboxylic acids, polyoxyalkylenated (C₆-C₂₄)alkylamido ether carboxylic acids and their salts, for instance those comprising 2 to 50 ethylene oxide units, and their mixtures.

Further, alkyl sulphates, alkyl ether sulphates and alkyl ether carboxylates, and their mixtures, for example in the form of alkali metal, alkaline earth metal, ammonium, amine, or aminoalcohol salts may be used in accordance with the present disclosure.

The ionic surfactant can be, for example, a cationic surfactant.

Non-limiting examples of the non-ionic surfactants which can be used in the cosmetic composition as disclosed herein are well-known compounds (see, for example, “Handbook of Surfactants” by M. R. Porter, published by Blackie & Son (Glasgow and London), 1991, pp 116-178) and are chosen, for example, from polyethoxylated, polypropoxylated or polyglycerolated fatty alcohols, fatty α-diols, fatty (C₁-C₂₀)alkylphenols, or fatty acids comprising a fatty chain comprising, for example, 8 to 18 carbon atoms, it being possible for the number of ethylene oxide or propylene oxide groups to range from 2 to 50 and it being possible for the number of glycerol groups to range, for example, between 2 and 30.

Non-limiting mention may also be made of condensates of ethylene oxide and of propylene oxide with fatty alcohols; polyethoxylated fatty amides, for example, comprising 2 to 30 ethylene oxide units, polyglycerolated fatty amides comprising 1 to 5 glycerol groups and, for instance, from between 1.5 and 4, sorbitan ethoxylated fatty acid esters comprising 2 to 30 ethylene oxide units, sucrose fatty acid esters, polyethylene glycol esters of fatty acids, N—((C₆-C₂₄)alkyl)glucamine derivatives, or amine oxides, such as oxides of ((C₁₀-C₁₄)alkyl)amines or N—((C₁₀-C₁₄)acyl)aminopropylmorpholine oxides.

Among the non-ionic surfactants disclosed herein, non-limiting examples include polyethoxylated, polypropoxylated and polyglycerolated alcohols.

In at least one embodiment of the present disclosure, the surfactant or surfactants are present at a concentration ranging from 0.01 to 20% by weight, such as at a concentration between 0.05 and 10% by weight and, for example, at a concentration between 0.1 and 5% by weight, with respect to the total weight of the composition.

The cosmetic composition according to the disclosure can additionally comprise at least one cosmetic adjuvant chosen from conditioning agents of ester type, antimousseing agents, moisturizing agents, emollients, glycols, plasticizers, inorganic thickening agents, polymeric or nonpolymeric and associative or non-associative organic thickening agents, water-soluble or fat-soluble sunscreens which may or may not be of silicone nature, permanent or temporary colourings, fragrances, peptizing agents, preservatives, ceramides and pseudoceramides, vitamins and provitamins, including panthenol, proteins, sequestering agents, solubilizing agents, basifying agents, acidifying agents, corrosion inhibitors, reducing agents or antioxidants, oxidizing agents, inorganic fillers, and glitter.

A person skilled in the art will take care to choose the possible adjuvants and their amounts so that they do not harm the properties of the compositions of the present disclosure.

The cosmetic adjuvant or adjuvants can be present at a concentration ranging from, for instance, 0.001 to 50% by weight, with respect to the total weight of the composition.

As used herein, the term “cosmetically acceptable medium” is understood to mean a medium compatible with keratinous substances and, for example, the hair.

The cosmetically acceptable medium can be an alcoholic, aqueous, or aqueous/alcoholic medium. Thus, the medium can, for instance, be composed solely of water or of a monoalcohol or of a mixture of water and of at least one cosmetically acceptable monoalcohol, such as lower C₁-C₄ alcohols, polyol ethers exhibiting a free hydroxyl and their mixtures. The alcohol can be, for example, ethanol.

The present disclosure also relates to a method for the cosmetic treatment of the hair, for example for styling, comprising applying an effective amount of a composition, as disclosed herein, to dry or wet hair and in rinsing or in not rinsing, after an optional setting time or after an optional drying.

In one embodiment, the composition is a leave-in composition.

The present disclosure also relates a method of fixing keratin fibers, such as the hair, comprising applying the cosmetic composition to keratin fibers.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements.

The following examples are intended to illustrate the disclosure in a non-limiting manner.

### EXAMPLES

The following compositions according to the disclosure were prepared. The data below are expressed as % by weight of active material, with respect to the total weight of the composition.

Styling mousses

| | 1 | 2 |
|---|---|---|
| Lambda-Carrageenan | 1% | 1.2% |
| (Welgeenan ED 1039, sold by Eurogum) | | |
| APG (Plantacare 2000 UP, sold by Cognis) | 1.7% | 1.3% |
| Ionic or non-ionic surfactant (Polysorbate-20) | 0.75% | — |
| Non-ionic fixing polymer (PVP) | — | 3% |
| Denatured alcohol | — | 5% |
| Propellent gas | 3.5% | 3.5% |
| (hydrocarbons, CO₂, nitrous oxide, and the like) | | |
| Preservatives, neutralizing agent, fragrance | q.s. | q.s. |
| Water q.s. for | 100% | 100% |

This composition, incorporated in an aerosol device, generated a quality mousse which conferred a very good treating effect on wet hair.

Styling gels

| | 1 | 2 |
|---|---|---|
| Lambda-Carrageenan | 3.5% | 4% |
| (Satiagum UTC 10, sold by Degussa) | | |
| APG (Oramix NS 10, sold by Seppic) | 0.6% | 0.8% |
| Ionic or non-ionic fixing polymer (PVP) | — | 2.5% |
| Denatured alcohol | — | 5% |
| Preservatives, neutralizing agent, fragrance | q.s. | q.s. |
| Water q.s. for | 100% | 100% |

This gel, applied to wet hair, was not very tacky. The fixing level was highly satisfactory.

Conditioners

| | 1 | 2 |
|---|---|---|
| Lambda-Carrageenan | 3.2% | 3% |
| (Welgeenan ED 1039, sold by Eurogum) | | |
| APG (Plantacare 2000 UP, sold by Cognis) | 0.9% | 1% |
| Non-ionic fixing polymer (copolymer 845) | — | 3% |
| Denatured alcohol | — | 5% |
| Silicone (dimethicone) | 2% | — |
| Cations (cetrimonium chloride) | — | 0.5% |
| Preservatives, neutralizing agent, fragrance | q.s. | q.s. |
| Water q.s. for | 100% | 100% |

This gel, applied to wet hair, was not very tacky and resulted in quality blow drying.

What is claimed is:

1. A cosmetic composition for the treatment of keratinous fibers, comprising, in a cosmetically acceptable medium:
at least one polysaccharide chosen from lambda-carrageenans, and
at least one non-ionic surface-active agent chosen from alkylpolyglucosides, wherein the polysaccharide chosen from lambda-carrageenans is present in an amount between 0.1 and 30% by weight of the total weight of the composition.

2. A composition according to claim 1, wherein the at least one non-ionic surface-active agent chosen from alkylpolyglucosides is a compound of following formula (I):
R₁—O—(R₂—O)ₐ-(L)_{b} (I)
wherein
R₁ is chosen from linear or branched alkyl and/or alkenyl radicals comprising from 8 to 24 carbon atoms and from alkylphenyl radicals, the linear or branched alkyl group of which comprises from 8 to 24 carbon atoms,
R₂ is chosen from alkylene radicals comprising 2 to 4 carbon atoms,
L is chosen from a reduced sugar comprising from 5 to 6 carbon atoms,
a ranges from 0 to 10, and
b ranges from 1 to 15.

3. A composition according to claim 2, wherein the at least one non-ionic surface-active agent chosen from alkylpolyglucosides is a compound of formula (I) wherein
R₁ is chosen from linear or branched alkyl and/or alkenyl radicals comprising 9 to 14 carbon atoms,
a is zero,
L is glucose, and
b ranges from 1 to 4.

4. A composition according to claim 1, wherein the at least one non-ionic surface-active agent chosen from alkylpolyglucosides is present in the composition in an amount ranging from 0.05 to 30% by weight of the total weight of the cosmetic composition.

5. A composition according to claim 1, wherein the molecular weight (MW) of the at least one polysaccharide chosen from lambda-carrageenans ranges from 100,000 to 1,000,000 a.m.u.

6. A composition according to claim 5, wherein the molecular weight of the at least one polysaccharide ranges from 250,000 to 800,000 a.m.u.

7. A composition according to claim 1, wherein the at least one polysaccharide is present in the cosmetic composition in an amount ranging from 0.2 to 20% by weight with respect to the total weight of the composition.

8. A composition according to claim 7, wherein the at least one polysaccharide is present in the cosmetic composition in an amount ranging from 0.5 to 15% by weight with respect to the total weight of the composition.

9. A composition according to claim 1, additionally comprising at least one additive chosen from a silicone, a fatty substance, a fixing polymer, and a surfactant.

10. A composition according to claim 1, additionally comprising at least one cosmetic adjuvant chosen from conditioning agents chosen from esters, antimousseing agents, moisturizing agents, emollients, glycols, plasticizers, inorganic thickening agents, polymeric or nonpolymeric and associative or non-associative organic thickening agents, water-soluble or fat-soluble sunscreens which may or may not be of silicone nature, permanent or temporary colorings, fragrances, peptizing agents, preservatives, ceramides and pseudoceramides, vitamins and provitamins, proteins, sequestering agents, solubilizing agents, basifying agents, acidifying agents, corrosion inhibitors, reducing agents or antioxidants, oxidizing agents, inorganic fillers, and glitter.

11. A composition according to claim 1, wherein the cosmetically acceptable medium is an aqueous, alcoholic or aqueous/alcoholic medium.

12. A cosmetic method for treating keratin fibers comprising
applying to the fibers a cosmetic composition comprising, in a cosmetically acceptable medium:
at least one polysaccharide chosen from lambda-carrageenans, and
at least one non-ionic surface-active agent chosen from alkylpolyglucosides, wherein the at least one polysaccharide chosen from lambda-carrageenans is present in an amount between 0.1 and 30% by weight of the total weight of the composition.

13. A cosmetic method according to claim 12, wherein the application of said composition is optionally followed by rinsing.

14. A cosmetic method of fixing keratinous fibers comprising
applying to the fibers a cosmetic composition comprising, in a cosmetically acceptable medium:
at least one polysaccharide chosen from lambda-carrageenans, and
at least one non-ionic surface-active agent chosen from alkylpolyglucosides, wherein the at least one polysaccharide chosen from lambda-carrageenans is present in an amount between 0.1 and 30% by weight of the total weight of the composition.

15. A cosmetic method of caring for keratinous fibers comprising
applying to the fibers a cosmetic composition comprising, in a cosmetically acceptable medium:
at least one polysaccharide chosen from lambda-carrageenans, and
at least one non-ionic surface-active agent chosen from alkylpolyglucosides, wherein the at least one polysaccharide chosen from lambda-carrageenans is present in an amount between 0.1 and 30% by weight of the total weight of the composition.
